(19) United States
(12) Patent Application Publication
(10) Pub. No.: US 2016/0143530 A1
(43) Pub. Date: May 26, 2016

(54) Smart Patient Monitoring

(71) Applicants: Mark A. Kijek, Joliet, IL (US); Jeffrey J. Williams, Shorewood, IL (US)

(72) Inventors: Mark A. Kijek, Joliet, IL (US); Jeffrey J. Williams, Shorewood, IL (US)

(21) Appl. No.: 14/950,506

(22) Filed: Nov. 24, 2015

Related U.S. Application Data
(60) Provisional application No. 62/083,505, filed on Nov. 24, 2014.

(51) Int. Cl. A61B 5/00; A61B 5/0402; A61B 5/0476

(52) U.S. Cl. CPC A61B 5/0015; A61B 5/7475; A61B 5/7278; A61B 5/0476; A61B 5/0402

(57) ABSTRACT

A patient monitoring method wherein data is received from a transducer by an electronic device, the electronic device receives input from the patient regarding timing of various events, the data is processed to incorporate the event timing data, and the data is then transmitted to a terminal. The terminal is accessible by, e.g., a healthcare provider. The transducer is used to perform medical diagnostic tests, such as fetal non-stress tests, electrocardiography, Holter monitoring, and electroencephalography. In the event that an emergency is identified, the healthcare provider can take immediate action. The utilization of an electronic device, e.g., a smartphone, by patients not only ensures ease of use and compliance with testing, but also conveniently allows patients to take an active role in their healthcare.

### CROSS REFERENCE TO RELATED APPLICATIONS

This application claims the benefit of U.S. Provisional Application No. 62/083,505 filed on Nov. 24, 2014. The above identified patent application is herein incorporated by reference in its entirety to provide continuity of disclosure.

### BACKGROUND OF THE INVENTION

The present invention relates to a patient monitoring method. More specifically, the present invention relates to receiving medical diagnostic data from a calibrated transducer and processing it with event inputs submitted by a user.

Many people agree that there are few things more important than health. As such, many individuals are willing to travel long distances and miss work in order to receive medical care. This not only increases the cost of health care for patients, but may also jeopardize job security and advancement opportunities. There are also many patients that are indirectly denied access to medical care because of transportation issues. These patients cannot comply with routine medical testing and often do not seek medical care at the onset of illness. Therefore, there exists a need for patients to have access to remote medical testing that can be transmitted to a healthcare provider in a timely fashion or in real-time.

There are existing devices that are capable of relaying information to monitoring systems utilizing wireless technology. However, some methods require patients to receive devices from and return them to their healthcare provider in person for interpretation. Other advanced devices exist where a transducer transmits monitoring information to a patient's personal computer using Bluetooth technology. The data must then be transmitted from the patient's personal computer to the healthcare provider via the internet. Thus, the recorded data must be in a format that is accepted by the healthcare provider's internet platform, or the user must manually enter the recorded results. Further, existing devices do not allow users to comment on and record symptoms experienced during the test.

Therefore, there is a need for a patient monitoring method that seamlessly transfers data from the user's smartphone to the healthcare provider's electronic device. Further, there is a need for method that requires recording the time at which the user experiences symptoms and correlating that with the data received from the transducer.

### SUMMARY OF THE INVENTION

In view of the foregoing disadvantages inherent in the known types of patient monitoring methods now present in the prior art, the present invention provides a method utilizing an electronic device wherein the user can conveniently conduct medical tests at home and transmit results to a healthcare provider. The present method comprises the steps of recording data collected by a transducer, receiving event input from a user, processing the recorded data with the event input, graphing the recorded data as a function of time to generate a data graph, graphing the event input as a function of time to generate an event graph, overlaying the data graph with the event graph, and sending the results to a terminal. The transducer can communicate with an electronic device wirelessly or through a wired connection. The transducer can be utilized to monitor the well-being of a fetus, heart rhythms, brain activity, and the like.

The device can be utilized in one's home and enables users to have remote medical care, wherein the information can be immediately transmitted to a terminal via the electronic device. The electronic device will alert the user in the event immediate medical attention is required. In addition, a user accessing the terminal, such as a healthcare provider, can then respond to the patient.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a diagram illustrating one embodiment of a monitor device used for a fetal non-stress test.

FIG. 2 shows a diagram illustrating one embodiment for a cap used for an EEG.

FIG. 3 shows a flowchart illustrating an embodiment of a method for patient monitoring.

FIG. 4 shows a diagram illustrating an embodiment of pre-processed data.

FIG. 5 shows a diagram illustrating an embodiment of data post-processing.

### DETAILED DESCRIPTION OF THE INVENTION

Reference is made herein to the attached drawings. Like reference numerals are used throughout the drawings to depict like or similar elements of the patient monitoring system. For the purposes of presenting a brief and clear description of the present invention, the preferred embodiment will be discussed as used for recording medical diagnostics and transmitting the results to a terminal. The figures are intended for representative purposes only and should not be considered to be limiting in any respect.

As used herein, “processor” refers to one or more devices, circuits, and/or cores configured to process data, such as a set of steps according to a computer program. Unless stated otherwise, a component such as a processor or interface described as being configured to perform a task includes both components temporarily configured to perform a task at a specified time and components manufactured to perform a task. As used herein, “logic” refers to (i) logic implemented as computer instructions and/or data within one or more computer processes and/or (ii) logic implemented in electric circuitry. Unless stated otherwise, the patient monitoring system utilizes multiple languages and is not limited to English. As used herein, “healthcare provider” is any individual or entity that provides medical services. As used herein, “transducer” refers to any device that emits a signal from sound waves or electrical impulses. An example of such a device is an ultrasound transducer capable of converting ultrasound waves into electrical signals, or vice versa.

The present invention is a patient monitoring method. The purpose of the present invention is to enable a patient to complete medical diagnostic tests, record and process the resulting data, and transfer the results to a terminal, which may be accessed by a healthcare provider. The present invention can be used to perform medical diagnostic tests such as fetal non-stress tests, electrocardiography (EKG), Holter monitoring, and electroencephalography (EEG).

The patient monitoring method is most conveniently performed utilizing an electronic device and thus that embodiment will be predominantly discussed. On example of such electronic device is a smartphone. The method involves first, recording data collected from the transducer; second, receiving event input from the user; third, processing and correlating the recorded data with the event input; fourth, graphing the recorded data as a function of time to generate a data graph; fifth, graphing the event input as a function of time to generate an event graph; sixth, overlaying the data graph with the event graph; and finally, transmitting the data graph to a terminal.

Before a user can perform the patient monitoring method, the user must first place a transducer over the region of the body to be monitored. For example, if the diagnostic test is an EEG, a cap containing transducers that measure the electrical impulses of the brain could be secured to the head of the user. If the test is a fetal non-stress test, the user could strap a transducer capable of generating sound waves around her abdomen. If the test is an EKG or Holter monitoring, the user could wear a device that connects leads to the body containing transducers that measure the electrical impulses of the heart.

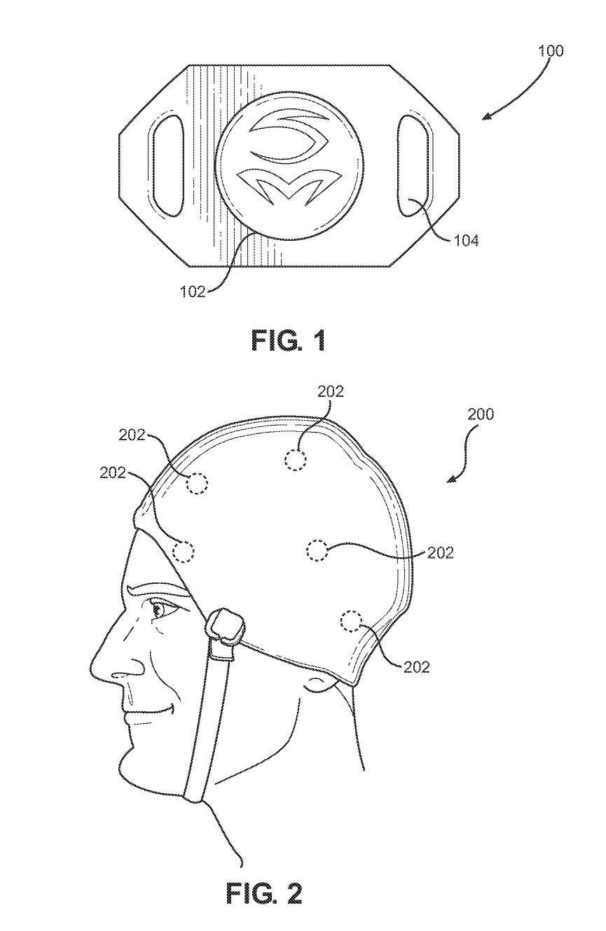

Referring now to FIG. 1, there is shown a diagram illustrating one embodiment of a monitor device used for a fetal non-stress test. In the depicted embodiment, a monitor device 100 contains a transducer 102 that emits sound waves. The user secures the monitor device 100 against her abdomen by looping and hooking straps through an aperture 104 in the monitor device 100. This will allow the transducer 102 to remain in a fixed position firmly pressed against the user's abdomen during the test. In various embodiments, the monitor device 100 is wirelessly connected to the electronic device. In other embodiments, the monitor device 100 is connected to the electronic device via a wired connection.

Referring now to FIG. 2, there is shown a diagram illustrating one embodiment for a cap used for an EEG. In the depicted embodiment, a cap 200 is outfitted with multiple transducers 202. The transducers 202 in the cap 200 will measure electrical impulses of the brain and transmit corresponding signals. In various embodiments, the cap 200 is wirelessly connected to the electronic device. In other embodiments, the cap 200 is connected to an electronic device via a wired connection.

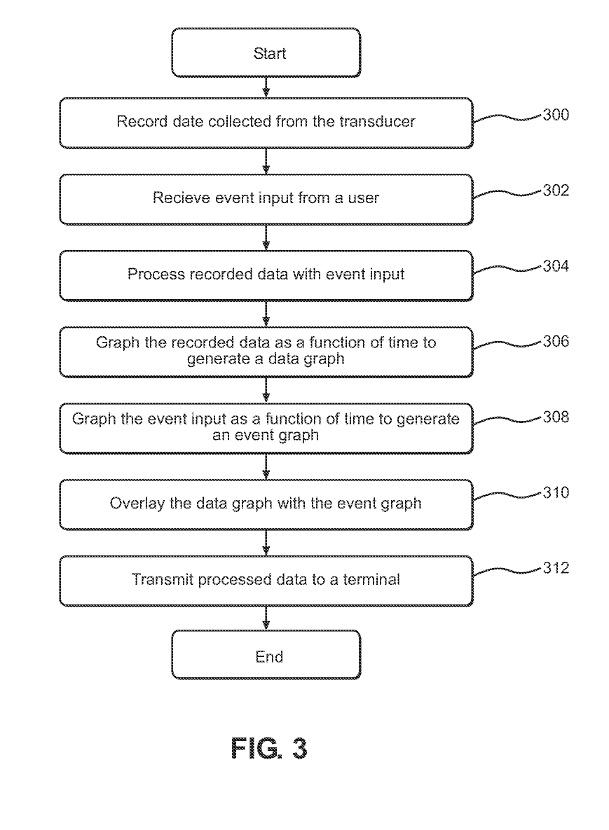

Once a transducer is secured to the region of the body that is being tested and is calibrated, the user can perform the method of the present invention. Referring now to FIG. 3, there is shown a flowchart illustrating an embodiment of a method for patient monitoring. At 300, once the user has selected to begin the diagnostic test, data is received from the transducer and recorded. When the user selects to begin the test, the start date and time will be automatically recorded. The diagnostic test will run for a predetermined time, although the user may select to end the test earlier. Similarly, the date and time the test is terminated will be automatically recorded.

At 302, event input is received from the user throughout the diagnostic test. While the test is ongoing, the logic receives input from the user as to the occurrence of an event. Each time the logic receives an event input, the logic automatically tags that event input with the date and time it is received. In one embodiment, the user can click a button corresponding to the pre-programmed event he or she is experiencing. For example, if the diagnostic test is a fetal non-stress test and the transducer is measuring the heart rate of the fetus, the user can click an event button every time she feels the fetus move. In another embodiment, the user can click an event button at any point during the diagnostic test and a text box will appear. The user can type an explanation into the text box about the symptoms he or she is experiencing. For example, if the diagnostic test is an EEG, the user might click the event button and type: “having severe frontal headache.” In another example, if the diagnostic test is an EKG or Holter monitoring, the user might click the event button and type: “shortness of breath.”

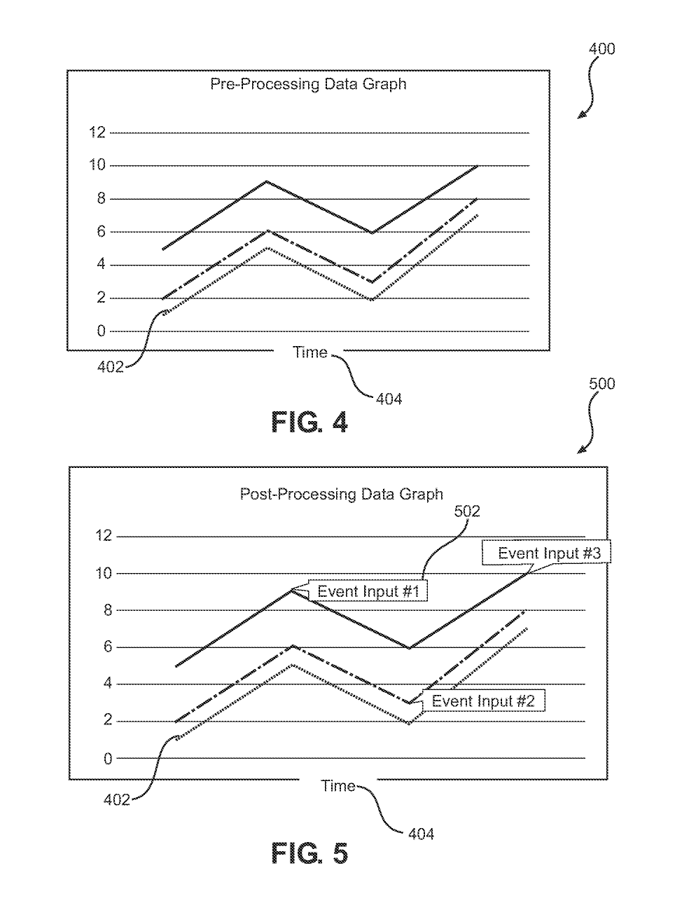

At 304, when the test is complete, the recorded data is processed and correlated with the event input received. At 306, the recorded data is graphed as a function of time to generate a data graph. For example, as shown in the embodiment depicted in FIG. 4, the data 402 received from the transducer is graphed along a time axis 404. At 308, the event input received by the logic is also graphed as a function of time to generate an event graph.

At 310, the data graph is overlaid with the event graph. For example, as shown in the embodiment depicted in FIG. 5, the tagged events 502 overlay the data graph 400 to produce a processed data graph 500. The processing and correlation of the signal data with the event inputs allow the physician to associate events with the data received from the diagnostic test. Using this association, the physician can get a better overall picture of the patient's health, which can aid tremendously in diagnosis and treatment. For example, physicians can account for irregularities in data, establish baseline activity levels, or determine a biological explanation for the occurrence of an event.

Finally, at 312, a terminal receives the processed diagnostic test results. The processed and correlated data is transmitted to a terminal. In various embodiments, this terminal is accessible by a physician or other healthcare provider. In some embodiments, the terminal will also receive an accompanying log of the tagged events with a description of symptoms experienced by the patient. Quick transmission of the diagnostic test results will allow physicians to provide faster diagnosis and treatment plans. This is especially important if the data shows that the user in need of emergency care.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A patient monitoring method, comprising the steps of:
recording signals collected from a transducer;
receiving an event input from a user;
processing recorded data with the event input;
graphing the recorded data as a function of time to generate a data graph;
graphing the event input as a function of time to generate an event graph;
overlaying the data graph with the event graph; and
transmitting the data graph to a terminal.

2. The method of claim 1, further comprising the step of tagging the event input with a date and a time.

3. The method of claim 1, wherein the transducer is configured to receive electrical impulses from a brain.

4. The method of claim 1, wherein the transducer is configured to receive electrical impulses from a heart.

5. The method of claim 1, wherein the transducer is configured to generate sound waves.

6. The method of claim 1, wherein the event input includes a list of user-selectable events.

7. A non-transitory computer-readable medium storing a logic that, when executed by a processor of a computer, causes the computer to perform a method comprising the steps of:
recording data collected from the transducer;
receiving an event input from a user;
processing recorded data with the event input;
graphing the recorded data as a function of time to generate a data graph;
graphing the event input as a function of time to generate an event graph;
overlaying the data graph with the event graph; and
transmitting processed data to a terminal.

8. The method of claim 7, further comprising the step of tagging the event input with a date and a time.

9. The method of claim 7, wherein the transducer is configured to receive electrical impulses from a brain.

10. The method of claim 7, wherein the transducer is configured to receive electrical impulses from a heart.

11. The method of claim 7, wherein the transducer is configured to generate sound waves

12. The method of claim 7, wherein the event input includes a list of user-selectable events.

13. A computer device with memory stored instructions which, when executed by a processor, causes the processor to perform the method comprising the steps of:
recording data collected from the transducer;
receiving an event input from a user;
processing recorded data with the event input;
graphing the recorded data as a function of time to generate a data graph;
graphing the event input as a function of time to generate an event graph;
overlaying the data graph with the event graph; and
transmitting processed data to a terminal.

14. The method of claim 13, further comprising the step of tagging the event input with a date and a time.

15. The method of claim 13, wherein the transducer is configured to receive electrical impulses from a brain.

16. The method of claim 13, wherein the transducer is configured to receive electrical impulses from a heart.

17. The method of claim 13, wherein the transducer is configured to generate sound waves.

18. The method of claim 13, wherein the event input includes a list of user-selectable events.
